(19) United States
(12) Patent Application Publication
(10) Pub. No.: US 2026/0354800 A1
(43) Pub. Date: Nov. 12, 2026

(54) SYNTHETIC IMAGE GENERATION AND DISPLAY VIA IMAGING DEVICE

(71) Applicant: GE Precision Healthcare LLC, Waukesha, WI (US)

(72) Inventors: Anuprriya Gogna, Bengaluru (IN); Edina Timkó, Budapest (HU); Vanda Czipczer, Budapest (HU)

(73) Assignee: GE Precision Healthcare LLC, Waukesha, WI (US)

(21) Appl. No.: 19/205,568

(22) Filed: May 12, 2025

(51) Int. Cl. A61B 8/00; G06T 11/00; G06V 10/74; G06V 10/774; G06V 10/82; G16H 30/40

(52) U.S. Cl. CPC A61B 8/5215; A61B 8/461; G06T 11/00; G06V 10/761; G06V 10/774; G06V 10/82; G16H 30/40; G06T 2210/41; G06V 2201/03

(57) ABSTRACT

Systems and methods for displaying synthetically generated images on an imaging device are described herein. In one example, a system includes a processing circuit including a processor coupled to a memory device storing instructions. The instructions, when executed, cause the processing circuit to perform operations including acquiring a medical image obtained using an imaging device; identifying, from a database, one or more similar images based on the medical image; and generating, using a generative artificial intelligence model, synthetic image data using the one or more similar images such that the synthetic image data matches at least one characteristic of the medical image.

### FIELD

Embodiments of the subject matter disclosed herein relate to ultrasound imaging, and more particularly, to generating synthetic ultrasound data.

### BACKGROUND

During a medical imaging scan, a plurality of medical images of a patient are obtained to measure or detect various aspects of anatomical features present within the medical images. Furthermore, the medical images of the patient may be compared to reference images depicting the same anatomical features to determine whether the medical images of the patient depict an abnormality in the anatomical features.

### SUMMARY

An embodiment relates to an ultrasound imaging system. The ultrasound imaging system includes a transducer, a matching layer, a damping block, a display screen, and a processing circuit. The transducer is configured to transmit and receive an ultrasound signal. The matching layer is configured to have an acoustic impedance match between a tissue to be imaged and a material of the transducer. The damping block is configured to absorb ultrasound energy. The processing circuit includes a processor coupled to a memory device storing instructions thereon that, when executed, cause the processing circuit to perform operations. The operations include acquiring an ultrasound image obtained using the transducer. The operations include identifying, from a database, one or more similar images based on the ultrasound image. The operations include generating, using a generative artificial intelligence model, synthetic image data using the one or more similar images, such that the synthetic image data matches at least one characteristic of the ultrasound image.

Another embodiment relates to a system. The system includes a processing circuit including a processor coupled to a memory device storing instructions thereon that, when executed, cause the processing circuit to perform operations. The operations include acquiring a medical image obtained using an imaging device. The operations include identifying, from a database, one or more similar images based on the medical image. The operations include generating, using a generative artificial intelligence model, synthetic image data using the one or more similar images such that the synthetic image data matches at least one characteristic of the medical image.

Another embodiment relates to a method. The method includes acquiring, by a processing circuit of an ultrasound imaging system, an ultrasound image obtained from a transducer of the ultrasound imaging system. The method includes identifying, by the processing circuit and from a database, one or more similar images based on the ultrasound image. The method includes generating, by the processing circuit and using a generative artificial intelligence model, synthetic image data using the one or more similar images, such that the synthetic image data matches at least one characteristic of the ultrasound image. The method includes displaying, by the processing circuit and via a display screen of the ultrasound imaging system, the synthetic image data to a user.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a block diagram of an ultrasound imaging system, according to an example embodiment.

FIG. 2 is a block diagram of a processing circuit of the ultrasound imaging system of FIG. 1, according to an example embodiment.

FIG. 3 is a diagram illustrating synthetic image generation using a reference database, according to an example embodiment.

FIG. 4 is a diagram illustrating feature extraction performed during synthetic image generation, according to an example embodiment.

FIG. 5A is an ultrasound image obtained during an ultrasound procedure, according to an example embodiment.

FIG. 5B is a reference image, according to an example embodiment.

FIG. 5C is a synthetic ultrasound image, according to an example embodiment.

FIG. 6A is another ultrasound image obtained during an ultrasound procedure, according to an example embodiment.

FIG. 6B is another reference image, according to an example embodiment.

FIG. 6C is another synthetic ultrasound image, according to an example embodiment.

FIG. 7 is a flow chart illustrating a method for displaying synthetic image data during an ultrasound procedure, according to an example embodiment.

FIG. 8 is a flow chart illustrating a method for identifying reference images during the method of FIG. 7, according to an example embodiment.

FIG. 9 is a flow chart illustrating a method for generating the synthetic image data during the method of FIG. 7, according to an example embodiment.

### DETAILED DESCRIPTION

Referring generally to the figures, systems and methods for displaying synthetically generated images on an imaging device are disclosed. More specifically, the systems and methods described herein include acquiring an ultrasound image obtained by an imaging device, identifying one or more reference images that are similar to the acquired ultrasound image, generating synthetic image data using the reference images such that the synthetic image data matches at least one characteristic of the acquired ultrasound image, and displaying the synthetic image data to a user via the imaging device.

Generally, human anatomies are widely variable such as across shape, size, presence/absence of structures (e.g., structures owing to pathologies, etc.). As a result, identifying an abnormality within a medical image may be difficult, as the sonographer or other medical professional tries to determine whether a feature depicted in the image is due to a unique anatomy or to abnormality within the anatomy. During screening examinations, such as second trimester fetal examination or ovarian reserve assessment, amongst others, clinicians try to identify possible pathologies and differentiate the normal from the abnormal. In some cases, it may be helpful for the clinician to have anatomical sketches or reference images available at the time of examination so that the clinician, especially less experienced clinicians, can compare the patient scans with the standard views/reference images.

In existing systems, some reference images may be made available from literature during an imaging procedure. Such reference images currently available, however, may not visually correlate well with the medical images being obtained during the imaging procedure. That is, the reference images may differ from the images obtained during the imaging procedure because of variations in probe, acquisition settings, patient characteristics, scanner versions, and so on. Furthermore, in the existing systems, the reference images are not easily accessible to the clinician via the imaging device during the imaging procedure. Although reference images may be displayed in some cases, the reference images are static and do not align with the characteristics of the current images being obtained during the imaging procedure.

The systems and methods described herein, however, provide a technical solution to existing systems by replacing standard (e.g., currently available) reference images with synthetically generated (e.g., using artificial intelligence) reference images/datasets. These artificial intelligence generated images are generated to mimic the image characteristic and properties of the current scan, while preserving the underlying anatomical structures of a standard, normal image. Such a synthetic reference image provides better correlation and easier comparison of the current scan with the normal/standard anatomical visualization. The same can be extended to known or defined pathologies to assist the user during screening examinations. For example, a synthetic reference image may depict a cyst of a known size (e.g., from a previous imaging scan) such that the clinician can determine whether the cyst has grown by comparing current images to the synthetic image depicting the known size of the cyst.

This disclosure refers to a novel and adaptive mechanism for providing reference images/datasets to a clinician during a screening examination, which best mimic or match the image characteristic of the current acquisition/scan. Beneficially, the systems and methods described herein assist users, especially less experienced users, in comparing a current scan with a corresponding normal case. In some instances, as mentioned above, this feature can be further extended to provide reference images for known and well-defined pathological cases. Such systems and methods as described herein alleviate the need for a user to be highly experienced to remember relevant anatomical details while providing easy access to relatable images on the scanner itself. In this way, the user benefits from having visualization of relatable reference images on the scanner itself without manual intervention or requiring an explicit search. Therefore, the systems and methods described herein facilitate a quicker and more reliable diagnosis of the normalcy of the examination, thereby improving efficiency and reliability of the procedure.

The implementations described herein address a technical problem by providing enhanced data integration and analysis capabilities, which deliver a particular technical solution that streamlines and refines imaging procedures and pathology detection. The systems described herein are implemented to facilitate pathology detection by automatically displaying synthetic medical images depicting normal anatomical features, but having the image characteristics of a current image being obtained during the imaging procedure. In this way, reference images are generated such that they match the current images except for any anatomical differences, which may indicate the presence of a pathology. Accordingly, this approach provides a specific technical improvement to various technical problems, including those set forth herein.

The systems described herein may also reduce processing power by performing various processing operations simultaneously to provide synthetic image data rather than performing a plurality of processing operations individually and consuming unnecessary processing power. That is, the systems and methods described herein eliminate the need for a clinician to manually set the reference images on the scanner. Rather, the systems and methods described herein can automatically detect matching image(s) from a stored database of standard images (e.g., images that depict healthy anatomies, known pathologies, etc.) by image retrieval, and subsequently generate relevant and matched reference images for displaying to the user. Therefore, the automatic generation and display of reference images that match images being obtained in real-time facilitates a more efficient imaging procedure.

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

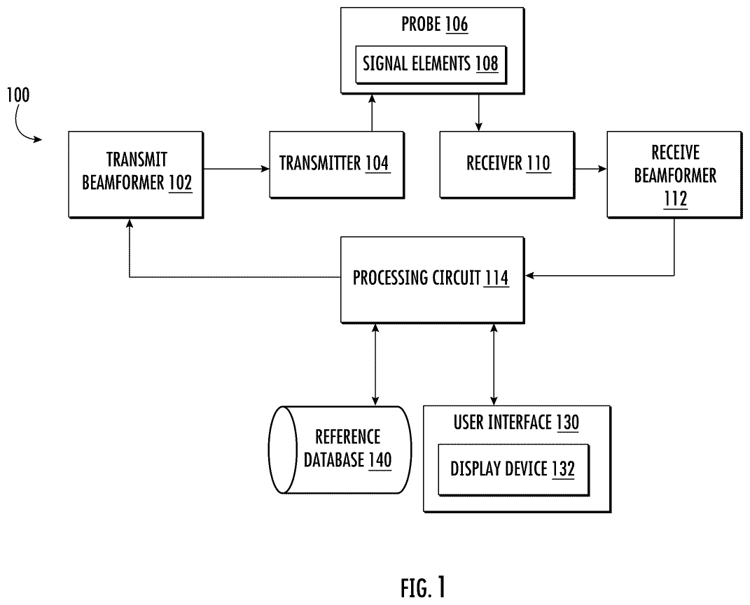

Referring to FIG. 1, a block diagram of an ultrasound imaging system 100 is shown. The ultrasound imaging system 100 may be used in a medical environment (e.g., hospitals, clinics, etc.), for example, by a sonographer, technician, or other clinician certified to collect ultrasound data from a patient. Although the systems and methods are described herein in the context of the ultrasound imaging system 100, it should be appreciated that the synthetic image generation and display described herein may be implemented using any of a variety of medical imaging systems (e.g., medical resonance imaging, x-ray, computed tomography, positron emission tomography, etc.).

An example of a procedure performed using the ultrasound imaging system 100 may be a fetal ultrasound. The fetal ultrasound may be a first trimester ultrasound, a second trimester ultrasound, a third trimester ultrasound, or any other ultrasound scan configured to monitor and assess fetal anatomy. In some embodiments, the fetal ultrasound may be configured to determine a due date, assess a placenta size and location, confirm a normal anatomy of the fetus, detect a heartbeat, and so on.

In at least one embodiment, the sonographer collects the ultrasound data by navigating a probe (e.g., probe 106, as described below) over the patient's uterus until a sufficient volume of ultrasound images are collected. The collected images are stored in a central storage device (e.g., memory 118) and analyzed by the sonographer. The sonographer generates a set of measurements from the images (e.g., 50-100 records), and the images and measurements are collectively reviewed by an obstetrician. The obstetrician provides any clinical findings/conclusions in a report submitted to the patient's medical record.

As shown in FIG. 1, the ultrasound imaging system 100 includes a transmit beamformer 102, a transmitter 104, a probe 106, a receiver 110, and a receive beamformer 112.

The transmit beamformer 102 may be either a hardware beamformer or a software beamformer. In embodiments where the transmit beamformer 102 is a hardware beamformer, the transmit beamformer 102 may include one or more of a graphics processing unit (GPU), a microprocessor, a central processing unit (CPU), a digital signal processor (DSP), or any other type of processor capable of performing logical operations. The transmit beamformer 102 may be configured to perform conventional beamforming techniques as well as techniques such as retrospective transmit beamforming (RTB). Alternatively, in embodiments where the transmit beamformer 102 is a software beamformer, a processor (e.g., processor 116, as described below) may be configured to perform some or all of the functions associated with the transmit beamformer 102.

The probe 106 may be a linear array probe, a curvilinear array probe, a sector probe, or any other type of probe configured to obtain two-dimensional (2D) B-mode data, 2D color flow data, M-mode data, three-dimensional (3D) data, four-dimensional (4D) data, or any other type of ultrasound data. Alternatively or additionally, the probe 106 may be any type of probe configured to obtain 2D B-mode data and data corresponding to another ultrasound mode that detects blood flow velocity in the direction of a vessel axis. In some embodiments, the probe 106 may include a position sensor configured to detect a position of the probe 106 relative to one or more reference locations. That is, the position sensor may continuously track movement (e.g., rotation, translation, orientation, etc.) of the probe 106 relative to the location of the probe 106 when the anatomy being imaged is identified. For example, the anatomy being imaged may be identified as a fetal skull at a first location of the probe 106. Then, the position sensor may track the movement of the probe 106 relative to the fetal skull in order to identify successive locations of the probe 106. In some embodiments, the position sensor may transmit position data to be stored within the ultrasound imaging system 100 (e.g., in memory 118).

The probe 106 may include a transducer configured to transmit and receive an ultrasound signal. In some embodiments, as shown in FIG. 1, the probe 106 includes signal elements 108. The signal elements 108 may be arranged in a transducer array, and in some embodiments may be arranged in a one-dimensional (1D) or 2D array. The transmit beamformer 102 and the transmitter 104 drive the signal elements 108 to emit pulsed ultrasonic signals into a body of a subject (e.g., a patient). For example, during a fetal examination, a sonographer or other clinician may navigate the probe 106 proximate to a patient's uterus so that the signal elements 108 in the probe 106 emit the pulsed ultrasonic signals into the patient's uterus. The pulsed ultrasonic signals are then back-scattered from anatomical structures in the body, such as blood cells or muscular tissues, to produce echoes that return to the signal elements 108. That is, the signal elements 108 may include the transducer configured to transmit and receive the ultrasound signal, a matching layer configured to have an acoustic impedance match between a tissue to be imaged and a material of the transducer (e.g., such that the pulsed electronic signals can be back-scattered from the anatomical structures in the body and received as echoes by the signal elements 108), and a damping block configured to absorb ultrasound energy.

The receiver 110 receives the echoes from the probe 106 and converts the echoes into electrical signals. The electrical signals are then passed through the receive beamformer 112, which produces the ultrasound data from the electrical signals. As described above with reference to the transmit beamformer 102, the receive beamformer 112 may be either a hardware beamformer or a software beamformer. In embodiments where the receive beamformer 112 is a hardware beamformer, the receive beamformer 112 may include one or more of a GPU, a microprocessor, a CPU, a DSP, or any other type of processor capable of performing logical operations. The receive beamformer 112 may be configured to perform conventional beamforming techniques as well as techniques such as retrospective transmit beamforming (RTB). Alternatively, in embodiments where the receive beamformer 112 is a software beamformer, a processor (e.g., processor 116, as described below) may be configured to perform some or all of the functions associated with the receive beamformer 112.

Although the transmit beamformer 102, the transmitter 104, the receiver 110, and the receive beamformer 112 are shown in FIG. 1 as being components of the ultrasound imaging system 100 that are distinct from the probe 106, it should be appreciated that in some embodiments, the probe 106 may include electronic circuitry configured to perform the functions of each of the transmit beamformer 102, the transmitter 104, the receiver 110, and/or the receive beamformer 112. That is, all or part of the transmit beamformer 102, the transmitter 104, the receiver 110, and/or the receive beamformer 112 may be situated within the probe 106.

Referring still to FIG. 1, the ultrasound imaging system 100 is shown to include a processing circuit 114. While shown as being separate from the probe 106 in FIG. 1, it will be appreciated that the processing circuit 114 can be part of the probe 106. For example, the processing circuit 114 can be disposed in a handheld housing of the probe 106 (e.g., in the case of the probe 106 being a wireless probe).

The ultrasound imaging system 100 is also shown to include a user interface 130. The user interface 130 may be used by a sonographer or other clinician to control operation of the ultrasound imaging system 100. For example, the sonographer may use the user interface 130 to control the input of patient data, to change a scanning or display parameter, to adjust a segmentation of an anatomical feature depicted in an ultrasound image, and/or to select various other modes, operations, parameters, etc. of the ultrasound imaging system 100. In some embodiments, the user interface 130 may include an off-the-shelf consumer electronic device such as a smartphone, a tablet, a laptop, and so on. For the purposes of this disclosure, the term “off-the-shelf consumer electronic device” is defined to be an electronic device that was designed and developed for general consumer use and one that was not specifically designed for use in a medical environment. Alternatively, in other embodiments, the user interface 130 may be an electronic device that was designed and developed for use in a medical environment.

According to some embodiments, the user interface 130 may be physically separate from the rest of the ultrasound imaging system 100 (e.g., the transmit beamformer 102, the transmitter 104, the probe 106, the receiver 110, the receive beamformer 112, or the processing circuit 114). The user interface 130 may communicate with the processing circuit 114 (e.g., processor 116) through a wireless protocol, such as Wi-Fi, Bluetooth, wireless local area network (WLAN), near-field communication, and so on. According to some embodiments, the user interface 130 may communicate with the processing circuit 114 through an application programming interface (API).

In some embodiments, the user interface 130 may include physical controls such as one or more of buttons, sliders, a rotary knob, a mouse, a keyboard, a trackball, hard keys linked to specific actions, soft keys that may be configured to control different functions, and so on. As shown in FIG. 1, the user interface 130 may also include a display device 132 (e.g., a display screen). In some embodiments, the display device 132 may be configured to display a graphical user interface (GUI) based on an instruction from a system memory (e.g., memory 118). The GUI may include user interface icons representing commands and instructions relating to the operation of the ultrasound imaging system 100. The user interface icons of the GUI may be configured such that a user (e.g., the sonographer, clinician, etc.) may select a specific user interface icon in order to initiate a specific function controlled by the GUI. For example, various user interface icons may be used to represent windows, menus, buttons, cursors, scroll bars, and so on. That is, the physical controls of the user interface 130 may be included as individual hardware elements, as user interface icons displayed on the display device 132, or as a combination of hardware elements and user interface icons.

In some embodiments, the display device 132 may include a touch-sensitive display device or a touch screen. According to such embodiments, the touch screen may be configured to interact with the GUI displayed by the display device 132 such that a user (e.g., the sonographer) can interact with the GUI via the touch screen. The touch screen may be a single-point touch screen that is configured to detect a single contact point at a time, or the touch screen may be a multi-point touch screen that is configured to detect multiple points of contact at a time. For embodiments where the touch screen is a multi-point touch screen, the touch screen may be configured to detect multi-point gestures involving contact from two or more of a user's fingers at a time. The touch screen may be a resistive touch screen, a capacitive touch screen, or any other type of touch screen that is configured to receive inputs from a stylus or one or more of a user's fingers. According to some embodiments, the touch screen may be an optical touch screen that uses technology such as infrared light or other frequencies of light to detect one or more points of contact initiated by a user. In some embodiments, the touch screen may be incorporated as part of the display device 132 or may be separate from the display device 132. The user interface 130 may also include a proximity sensor configured to detect objects and/or gestures that are within a predetermined distance (e.g., five feet, six inches, ten centimeters, etc.) of the proximity sensor. In various embodiments, the proximity sensor may be located on the display device 132 or as part of a touch screen that is separate from the display device 132.

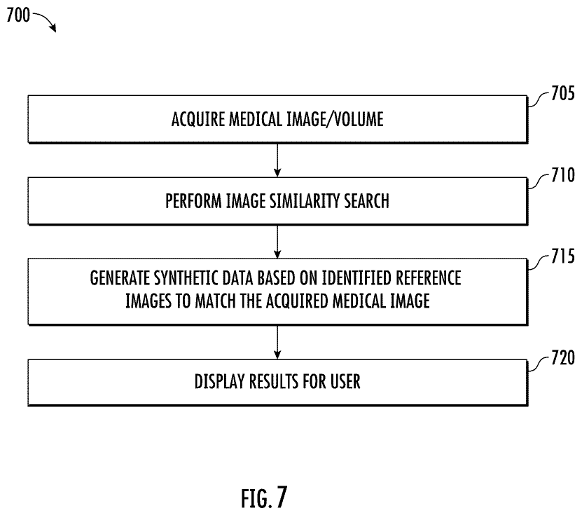

The ultrasound imaging system 100 may also include a reference database 140. The reference database 140 stores a variety of images or volumes obtained from multiple sources, having varying image qualities, acquired using multiple imaging machines, etc. As described herein, the reference database 140 may be a data source for generating deep learning models used to perform an image similarity search and synthetic style transfer (e.g., as described below with reference to FIGS. 7-9). In some embodiments, the reference database 140 refers to a database from which the processing circuit 114 (e.g., the image comparison circuit 122) may retrieve reference images (e.g., ultrasound images) during an ultrasound imaging procedure (e.g., during the image similarity search performed at step 715 of method 700, etc.). For instance, the reference database 140 may store reference image 410, reference image 610, etc., as described in greater detail below.

In some embodiments, the reference database 140 may be established by the ultrasound imaging system 100 using a plurality of reference images that are retrieved from a plurality of sources. For instance, the plurality of sources may include a plurality of image sources. Additionally or alternatively, the plurality of reference images stored in the reference database 140 may correspond to a plurality of image qualities (e.g., high amounts of noise, low amounts of noise, high resolution, low resolution, etc.). Furthermore, in some embodiments, the plurality of reference images may be obtained using a plurality of image devices (e.g., different models of ultrasound probes, different ultrasound imaging systems, etc.).

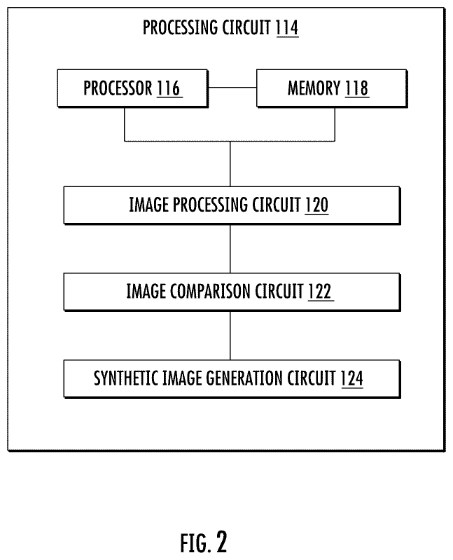

Referring to FIG. 2, the processing circuit 114 of the ultrasound imaging system 100 is shown in greater detail. As shown, the processing circuit 114 may include at least one processor 116, a memory 118, an image processing circuit 120, an image comparison circuit 122, and a synthetic image generation circuit 124. In this way, the processing circuit 114 may be structured or configured to execute or implement the instructions, commands, and/or control processes described herein with respect to the processor 116, the memory 118, the image processing circuit 120, the image comparison circuit 122, and the synthetic image generation circuit 124.

The processor 116 may include a CPU, a GPU, a microprocessor, a DSP, a general-purpose single- or multi-chip processor, a field-programmable gate array (FPGA), or any other type of processor capable of performing logical operations. A general-purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the processor 116 may be shared by multiple circuits (e.g., the circuits of the processor 116 may include or otherwise share the same processor which, in some example embodiments, may execute instructions stored, or otherwise accessed, via different areas of the memory 118). Alternatively or additionally, the processor 116 may be structured to perform or otherwise execute certain operations independent of one or more co-processors. In some embodiments, two or more processors may be coupled via a bus to enable independent, parallel, pipelined, or multi-threaded instruction execution. All such variations are intended to fall within the scope of the present disclosure.

The processor 116 may be configured to control the transmit beamformer 102, the transmitter 104, the receiver 110, and the receive beamformer 112. The processor 116 may also be in electronic communication with the probe 106. For purposes of this disclosure, the term “electronic communication” may be defined to include both wired and wireless communications.

In some embodiments, the processor 116 may be configured to control the probe 106 during data acquisition. That is, the processor 116 may control the data acquisition by controlling which of the signal elements 108 are active and by controlling a shape of the beam emitted from the probe 106. Alternatively or additionally, the processor 116 may include a complex demodulator configured to demodulate radio frequency (RF) data obtained by the probe 106 and generate raw data. According to other embodiments, the demodulation of the RF data may be performed by another component of the ultrasound imaging system 100. The processor 116 may perform the processing operations described herein according to a plurality of selectable ultrasound modalities.

Depending on a mode of operation of the ultrasound imaging system 100, the processor 116 may process ultrasound data obtained by the probe 106 according to the mode of operation to generate 2D or 3D image data. For example, the mode of operation may include B-mode, color flow Doppler mode, M-mode, color M-mode, spectral Doppler, elastography, TVI, strain, strain rate, and the like. Various of these modes of operation may be configured to, for instance, convert ultrasound data from beam space coordinates (e.g., received from the receive beamformer 112) to display space coordinates (e.g., such that the ultrasound data may be displayed as image data). In some embodiments, the mode of operation may allow for video processing by the processor 116 such that a series of images (e.g., processed ultrasound data) may be displayed in real-time while a scanning session/procedure is being performed on a patient. An operator of the ultrasound imaging system 100 (e.g., a sonographer) may switch between various modes in order to obtain a variety of ultrasound data and to perform a complete scan of an anatomical region of interest. For example, the operator may switch between modes using the user interface 130 (e.g., using physical controls, interface inputs representing physical controls, etc.). While the term “image” or “images” are used herein to for the purposes of example, it will be appreciated that such terms cover still images as well as videos, clips, or a series of images for each. For example, in some embodiments, the image or images may include a 1-2 second clip derived from the image data.

The processor 116 performs the processing operations in real-time as the echo signals are received by the receiver 110 from the probe 106. For the purposes of this disclosure, the term “real-time” is defined to include a procedure that is performed without any intentional delay. As an illustrative, non-limiting example, in certain instances, the ultrasound imaging system 100 may obtain images at a real-time volume-rate of 7-20 volumes/sec. It should be appreciated, however, that the real-time volume-rate may be dependent on the length of time that it takes to obtain each volume of data for display. Thus, the ultrasound imaging system 100 may be configured to obtain 2D data of an anatomical region at a faster rate than 3D data of the same anatomical region because it takes longer to obtain a volume of 3D data than the same volume of 2D data. Similarly, when the ultrasound imaging system 100 obtains a relatively large volume of data, the real-time volume-rate may be slower than for a smaller volume of data. For example, during an abdominal scan, the real-time volume-rate may be slower if the patient is an adult versus if the patient is an infant because the volume of data is larger for the adult than for the infant (e.g., due to the abdomen of an adult being larger than the abdomen of an infant). Therefore, certain implementations of the ultrasound imaging system 100 may have real-time volume-rates that are faster than 20 volumes/sec, while other implementations of the ultrasound imaging system 100 may have real-time volume-rates that are slower than 7 volumes/sec.

In some embodiments, the ultrasound imaging system 100 may include multiple processors configured to perform the processing operations/functionality described with reference to processor 116. For example, in such embodiments, a first processor of the multiple processors may be configured to demodulate and decimate the RF signal while a second processor of the multiple processors may be configured to further process the RF data prior to displaying an image representative of the data. It should be appreciated that other embodiments may use a different arrangement of processors.

The processor 116 may also be in electronic communication with the display device 132 such that the processor 116 may process ultrasound data obtained by the probe 106 and generate images to display on the display device 132.

As shown in FIG. 2, the processing circuit 114 also includes memory 118. The memory 118 may be configured to, for example, store processed volumes of data obtained by the ultrasound imaging system 100 (e.g., ultrasound data collected by the probe 106, user inputs received by the user interface 130, etc.). For example, the memory 118 may be a hospital picture archiving and communication system (PACS). The memory 118 (e.g., memory, memory unit, storage device, etc.) may include one or more devices (e.g., RAM, ROM, Flash memory, hard disk storage, etc.) for storing data and/or computer code for completing or facilitating the processes, layers, and modules described in the present application. The memory 118 may be or include tangible, non-transient volatile memory or non-volatile memory. The memory 118 may also include database components, object code components, script components, or any other type of information structure for supporting the activities and information structures described in the present application.

In various embodiments, the memory 118 may have varying capacity (e.g., storage space) across embodiments of the ultrasound imaging system 100. For example, the memory 118 may be configured to store at least 60 minutes' worth of ultrasound data. The ultrasound data may be stored in the memory 118 such that the ultrasound data may be retrieved according to an order/time of acquiring the data. That is, the ultrasound data may be stored with a timestamp indicating a time at which the ultrasound data was collected and may be retrieved starting with an oldest time at which the ultrasound data was collected.

The processing circuit 114 also includes the image processing circuit 120. The image processing circuit 120 is configured to receive image data obtained by the transducer of the probe 106 during an ultrasound scan. The image data refers to ultrasound data collected by the probe 106 while performing an ultrasound examination on a patient. For example, the image data may be collected during a fetal ultrasound and may therefore include various images of a patient's uterus and the fetal anatomy contained therein. The image processing circuit 120 may include multiple deep learning-based models configured to analyze the image data. For example, the image processing circuit 120 may be configured to identify a view from which the image data is captured, an anatomical structure or other feature captured by the image data, the presence of a pathology in the image data, and so on. The image processing circuit 120 may be configured to identify the anatomical structure using one or more algorithms (e.g., image processing algorithms such as edge detection, machine learning models, deep neural networks, etc.). In some embodiments, the image processing circuit 120 may identify anatomical features such as bones, blood vessels, organs, etc., based on a shape, relative proximity, apparent depth, orientation, etc. of said features in the image data.

As shown in FIG. 2, the processing circuit 114 may include the image comparison circuit 122. The image comparison circuit 122 is configured to compare ultrasound images obtained by the probe 106 to images stores in the reference database 140. That is, the image comparison circuit 122 may identify similar images from the reference database 140 using features identified by the image processing circuit 120, as described above. In some embodiments, the image comparison circuit 122 includes a self-supervised learning model. Beneficially, in such embodiments, the self-supervised learning model may be configured to learn global and local representations of images without using ground truth labeling.

The processing circuit 114 may include the synthetic image generation circuit 124. As described in greater detail herein, the synthetic image generation circuit 124 may be configured to generate synthetic image data using an ultrasound image obtained by the probe 106 and a reference image identified (e.g., by the image comparison circuit 122) from the reference database 140. In other words, the synthetic image generation circuit 124 may be configured to generate synthetic images that include standard anatomical features depicted in the reference image, while mimicking the style of the ultrasound image obtained by the probe 106. In some embodiments, the synthetic image generation circuit 124 includes a generative artificial intelligence model configured to generate the synthetic image data. For instance, the generative artificial intelligence model may be a generative adversarial network.

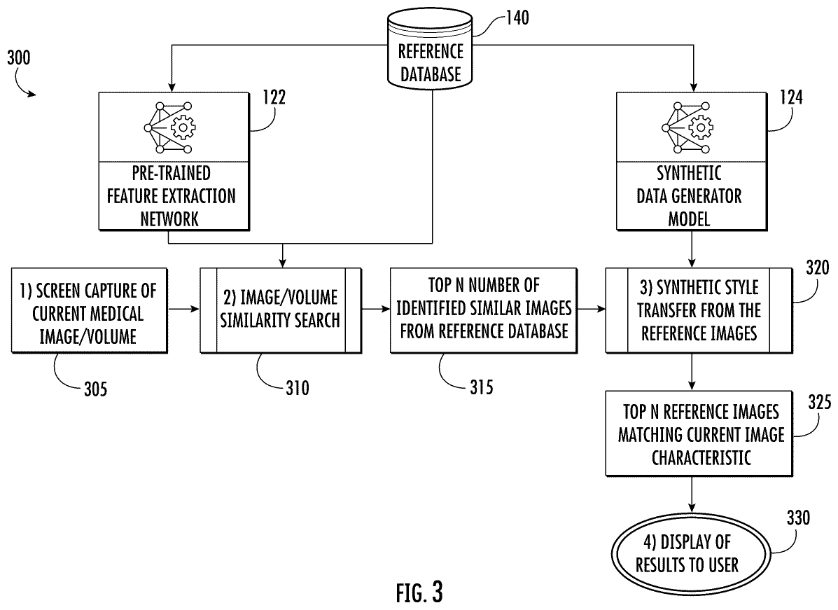

Referring to FIG. 3, a diagram illustrating a process 300 for reference image retrieval and style transfer is shown. The process 300 may include use of the image comparison circuit 122 (e.g., a pre-trained feature extraction network), the synthetic image generation circuit 124 (e.g., a synthetic data generator model), and the reference database 140. In some instances, process 300 may include establishing the reference database 140 by retrieving a plurality of reference images from a plurality of image sources, the plurality of reference images having a plurality of image qualities and having been obtained using a plurality of imaging devices. As shown in FIG. 3, the image comparison circuit 122 and the synthetic image generation circuit 124 are configured to access the reference database 140.

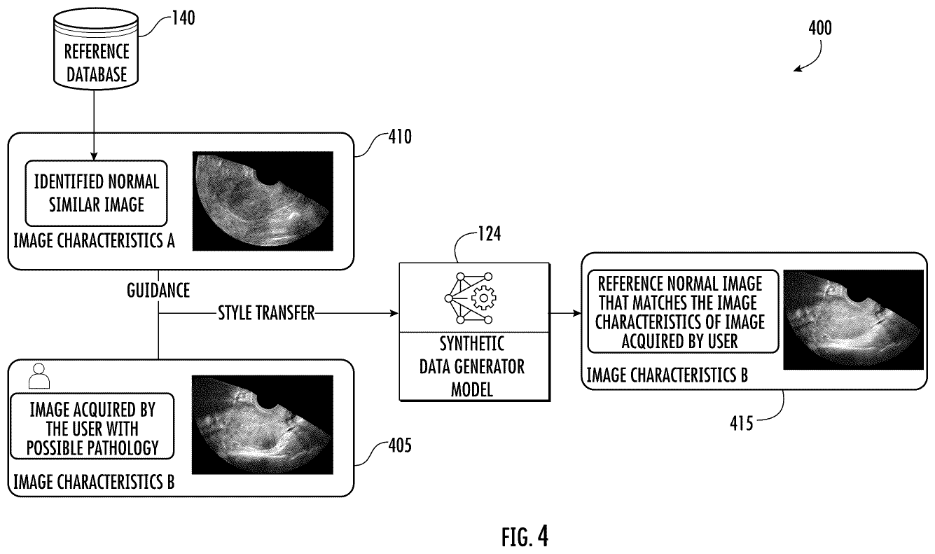

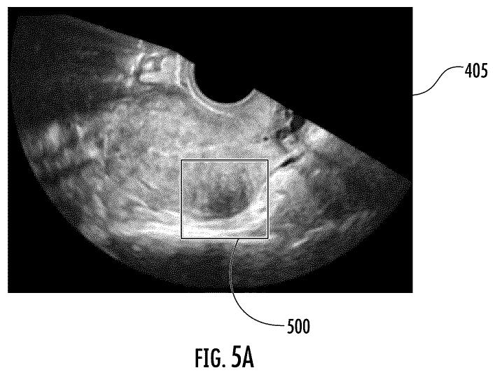

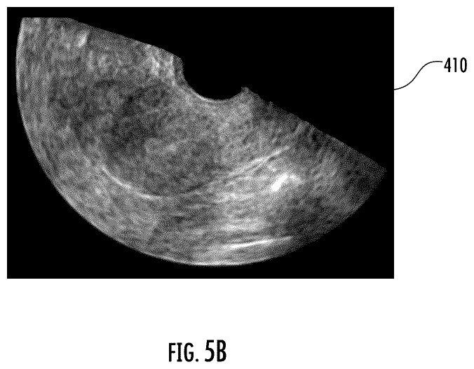

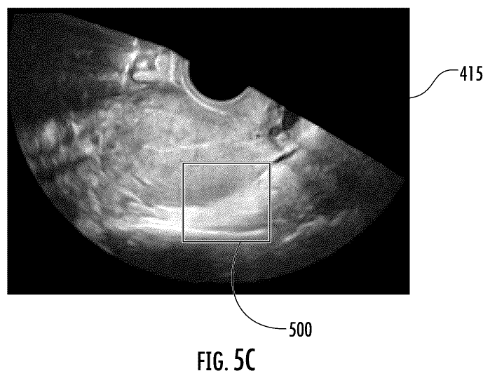

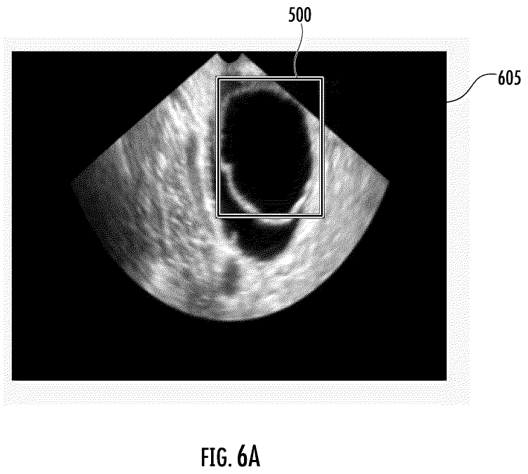

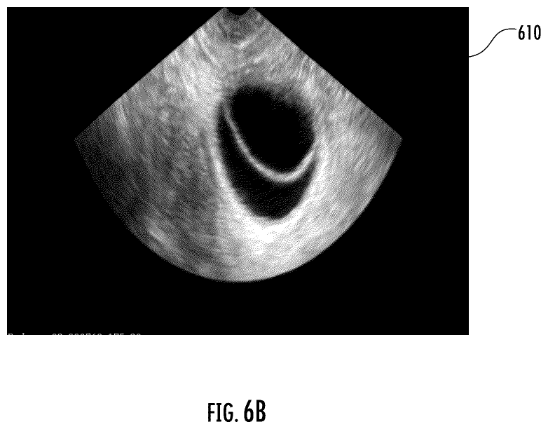

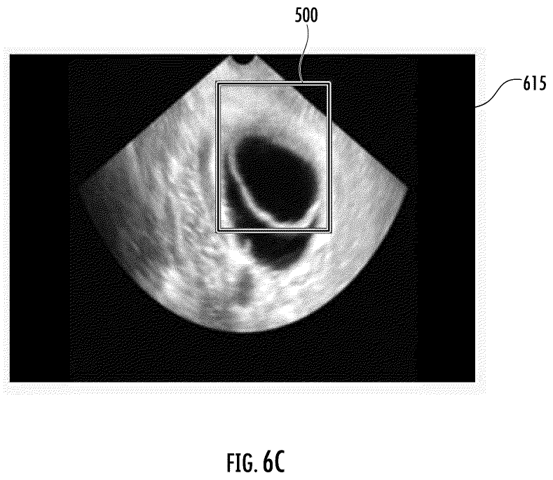

At step 305 of process 300, a screen capture of a current medical image is acquired. In some embodiments, the medical image may refer to an image volume. The current medical image or volume may be obtained using the transducer (e.g., probe 106) and may depict an anatomical region or feature of a patient. For example, the medical image or volume may depict an ovary. In some instances, the screen capture may be displayed on the display device 132 during an ultrasound imaging procedure. As an example, the medical image or volume may be ultrasound image 405 shown in FIGS. 4 and 5A or ultrasound image 605 shown in FIG. 6A.

Using the medical image from step 305, an image similarity search is performed at step 310. In some embodiments where the current medical image refers to an image volume, the image similarity search performed at step 310 may refer to an image volume similarity search.

During the similarity search, the image comparison circuit 122 is configured to identify similar images to the medical image acquired at step 305 from the reference database 140. For example, the similar images identified during the similarity search performed at step 310 may include reference image 410 shown in FIGS. 4 and 5B or reference image 610 shown in FIG. 6B. From the similarity search performed at step 310, as shown in FIG. 3, the N most similar images from the reference database 140 may be identified at step 315.

At step 320, the synthetic image generation circuit 124 may be configured to perform a synthetic style transfer from the reference images identified at step 315. In other words, the synthetic image generation circuit 124 is configured to generate synthetic image data from the identified reference images (e.g., from step 315) to match the image characteristic (e.g., style, etc.) of the current image (e.g., from step 305). As shown in FIG. 3, after the synthetic style transfer is performed by the synthetic image generation circuit 124 at step 320, the N most similar images from the reference database 140 match the current image characteristics (e.g., from step 305) at step 325. For instance, the resulting images provided at step 325 may include synthetic image 415 shown in FIGS. 4 and 5C or synthetic image 615 shown in FIG. 6C. Then, the results (e.g., the synthetic data generated by the synthetic image generation circuit 124) may be displayed to a user (e.g., sonographer, clinician, technician, medical professional, etc.) at step 330. In some embodiments, the results may be displayed via the display device 132. Furthermore, the results may be analyzed by the user to facilitate clinical assessment.

Referring to FIG. 4, a diagram illustrating process 400 for performing synthetic style transfer (e.g., step 320 of process 300) is shown. As shown, process 400 may begin with an ultrasound image 405 acquired by a user (e.g., sonographer, technician, clinician, etc.) depicting a possible pathology. Furthermore, the ultrasound image 405 may have image characteristics B. Then, a normal (e.g., without a possible pathology), similar image is identified from the reference database 140, shown as reference image 410. In some instances, the reference image 410 is identified by the image comparison circuit 122 based on reference image 410 depicting a same anatomical region or feature (e.g., uterus, ovary, etc.) as the ultrasound image 405. As shown, the reference image 410 may have image characteristics A.

Then, the reference image 410 may be used as guidance during the style transfer (e.g., transferring the style of ultrasound image 405 onto the reference image 410) performed by the synthetic image generation circuit 124. As shown, the synthetic image generation circuit 124 may be configured to generate synthetic image 415. More specifically, synthetic image 415 may depict the reference image 410 having image characteristics that match the image characteristics of the ultrasound image 405. For instance, where ultrasound image 405 has image characteristics B, the synthetic image 415 has image characteristics B.

Referring to FIGS. 5A-5C, the images from process 400 are shown in greater detail. That is, FIG. 5A shows the ultrasound image 405. In some embodiments, ultrasound image 405 may be obtained by a sonographer using the probe 106 during an ultrasound image procedure. The ultrasound image 405 depicts a uterus with a possible pathology 500. For instance, as shown in FIG. 5A, the possible pathology 500 may be a fibroid (e.g., an abnormal growth within the uterus).

FIG. 5B depicts the reference image 410 in greater detail. As described above, the reference image 410 may be identified by the image comparison circuit 122 from the reference database 140 based on the reference image 410 being most similar (e.g., depicting a same anatomical feature or region, etc.) to the ultrasound image 405. For example, as shown in FIG. 5B, the reference image 410 depicts a uterus. The reference image 410 does not, however, depict the possible pathology 500 (e.g., a fibroid). In this way, the reference image 410 may depict a normal, healthy, standard, etc. uterus.

Referring to FIG. 5C, the synthetic image 415 is shown. More specifically, the synthetic image 415 depicts the normal uterus of the reference image 410 shown in FIG. 5B, but has the image characteristics of the ultrasound image 405 shown in FIG. 5A. Furthermore, FIG. 5C illustrates that at the location of the possible pathology 500 (e.g., the fibroid shown in FIG. 5A), the synthetic image 415 does not depict the fibroid. In this way, a user (e.g., sonographer, clinician, technician, medical professional, etc.) may compare the uterus of the synthetic image 415 to the uterus of the ultrasound image 405 and may thereafter determine the presence of the possible pathology 500 (e.g., the fibroid) in the ultrasound image 405.

Referring to FIG. 6A, another example of an ultrasound image, shown as ultrasound image 605, is shown. In some embodiments, ultrasound image 605 may be the ultrasound image acquired during process 400, as described above. As shown, ultrasound image 605 depicts an ovary with the possible pathology 500. For instance, as shown in FIG. 6A, the possible pathology 500 may be an enlarged cyst (e.g., an abnormality within the ovary).

FIG. 6B depicts another example of a reference image, shown as reference image 610. In some embodiments, the reference image 610 may be the reference image identified during process 400, as described above. That is, the reference image 610 may be identified by the image comparison circuit 122 from the reference database 140 based on the reference image 610 being most similar (e.g., depicting a same anatomical feature or region, etc.) to the ultrasound image 605. For example, as shown in FIG. 6B, the reference image 610 depicts an ovary. The reference image 610 does not, however, depict the possible pathology 500 (e.g., the enlarged cyst). In this way, the reference image 610 may depict a normal, healthy, standard, etc. follicle.

Referring to FIG. 6C, another example of a synthetic image, shown as synthetic image 615, is shown. In some embodiments, the synthetic image 615 may be the synthetic image generated during process 400, as described above. That is, the synthetic image 615 may be generated by the synthetic image generation circuit 124 using the reference image 610, such that the synthetic image 615 matches at least one characteristic of the ultrasound image 605. More specifically, the synthetic image 615 depicts the ovary of the reference image 610 shown in FIG. 6B, but has the image characteristics of the ultrasound image 605 shown in FIG. 6A.

Furthermore, FIG. 6C illustrates that at the location of the possible pathology 500 (e.g., the enlarged cyst shown in FIG. 6A), the synthetic image 615 does not depict the enlarged cyst. In this way, a user (e.g., sonographer, clinician, technician, medical professional, etc.) may compare the uterus of the synthetic image 615 to the uterus of the ultrasound image 605 and may thereafter determine the presence of the possible pathology 500 (e.g., the fibroid) in the ultrasound image 605.

Referring to FIG. 7, a flow chart is shown illustrating a method 700 for displaying synthetic ultrasound data during an ultrasound imaging procedure. In at least one embodiment, the ultrasound imaging system referred to by method 700 is the ultrasound imaging system 100 described above with reference to FIGS. 1-2, and method 700 may be implemented by the ultrasound imaging system 100. In some embodiments, method 700 may be implemented as executable instructions in a memory of the ultrasound imaging system 100, such as the memory 118 of FIG. 2.

In some embodiments, method 700 may include establishing the reference database 140 using a plurality of reference images. As described above, the plurality of reference images may be retrieved from a plurality of image sources. Additionally or alternatively, the plurality of reference images may correspond to a plurality of image qualities. Furthermore, the plurality of reference images stored in the reference database 140 may be obtained using a plurality of imaging devices.

As shown in FIG. 7, method 700 may include acquiring a medical image or volume (e.g., an ultrasound image) at step 705. In some embodiments, step 705 may be performed in a similar or identical manner to step 305 of process 300, as described above with reference to FIG. 3. The medical image or volume acquired at step 705 may be obtained using the transducer of the ultrasound imaging system 100. For example, the medical image or volume acquired at step 705 may be ultrasound image 405 or ultrasound image 605.

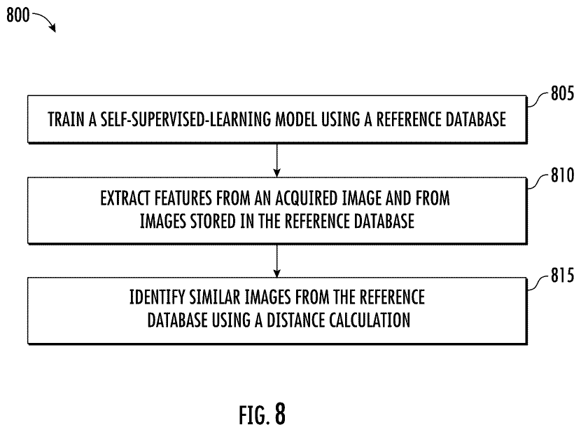

At step 710, a similarity search is performed. In some instances, step 710 may be performed in a similar or identical manner to step 310 of process 300, as described above with reference to FIG. 3. The similarity search may be performed at step 710 by the image comparison circuit 122. More specifically, the image comparison circuit 122 may be configured to identify, form the reference database 140, one or more similar images (e.g., reference images) based on the image acquired at step 705. For example, the similar images identified by the image comparison circuit 122 during the similarity search performed at step 710 may be reference image 410 or reference image 610. Furthermore, in some embodiments, the similar images may be identified by a self-supervised-learning model (e.g., as described in greater detail below with reference to FIG. 8).

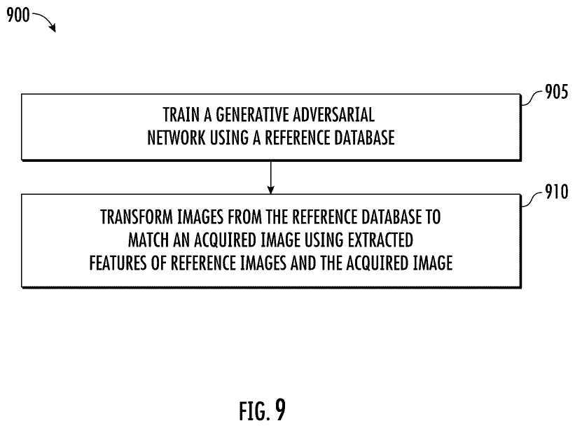

Then, method 700 may include generating synthetic data at step 715. More specifically, step 715 includes generating synthetic data based on the reference images identified during the similarity search performed at step 710 to match the medical image acquired at step 705. In other words, the synthetic data generated at step 715 may refer to synthetic image data generated by the synthetic image generation circuit 124 (e.g., a generative artificial intelligence model) using the one or more similar images identified at step 715, such that the synthetic image data matches at least one characteristic of the ultrasound image acquired at step 705. For example, the synthetic data generated at step 715 may be synthetic image 415 or synthetic image 615. As described above, the synthetic image generation circuit 124 may include a generative artificial intelligence model. Furthermore, in some instances, the generative artificial intelligence model may include a generative adversarial network (e.g., as described below with reference to FIG. 9). In some instances, step 715 may be performed in a similar or identical manner to step 320 of process 300, as described above with reference to FIG. 3.

At step 720, the results may be displayed to the user. That is, step 720 includes displaying the synthetic image data generated at step 715 to the user. In some embodiments, the synthetic image data may be displayed to the user via the display device 132. Furthermore, in some instances, step 720 may be performed in a similar or identical manner to step 330 of process 300, as described above with reference to FIG. 3.

Referring to FIG. 8, the similarity search performed at step 710 of method 700 is described in greater detail. That is, FIG. 8 depicts a method 800 for identifying the one or more similar images at step 710 of method 700. As shown, method 800 may include training a self-supervised-learning model (e.g., the image comparison circuit 122) using a reference database at step 805. For instance, the self-supervised-learning model may refer to the image comparison circuit 122. Additionally or alternatively, the reference database used to train the self-supervised-learning model may be the reference database 140, as described herein.

Using the trained self-supervised-learning model, step 810 may include extracting features (e.g., one or more characteristics, etc.) from an acquired image (e.g., an ultrasound image) and from images stored in the reference database (e.g., reference database 140). In some embodiments, the acquired image may refer to the medical image acquired at step 305 of process 300, at step 705 of method 700, etc. Additionally or alternatively, the acquired image may be ultrasound image 405 or ultrasound image 605, according to some embodiments.

Then, step 815 may include identifying similar images from the reference database using a distance calculation. That is, the self-supervised-learning model may be configured to identify at least one characteristic extracted from the acquired image. Then, the self-supervised-learning model may use a distance calculation (e.g., a cosine distance, etc.) between the at least one characteristic and each of a plurality of characteristics extracted from the images stored in the reference database. Based on the distance calculation between the extracted features, the N most similar images may be identified from the reference database.

Referring to FIG. 9, the synthetic data generation of step 715 during method 700 is described in greater detail. That is, FIG. 9 depicts a method 900 for generating the synthetic image data at step 715 of method 700. As shown, method 900 may include training a generative adversarial network (e.g., the synthetic image generation circuit 124) using a reference database (e.g., reference database 140) at step 905. In this way, the generative adversarial network may be trained using images having a plurality of different characteristics.

As shown in FIG. 9, step 910 may include transforming, using the generative adversarial network, images from the reference database to match an acquired image using extracted features of reference images and the acquired image. That is, step 910 may include using the N most similar images identified at step 815 of method 800 and the acquired image (e.g., ultrasound image 405, ultrasound image 605, etc.) as guidance for the generative adversarial network to transform/synthetize the reference images (e.g., reference image 410, reference image 610, etc.) to match the acquired image (e.g., current image).

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

1. An ultrasound imaging system comprising:
a transducer configured to transmit and receive an ultrasound signal;
a matching layer configured to have an acoustic impedance match between a tissue to be imaged and a material of the transducer;
a damping block configured to absorb ultrasound energy;
a display screen; and
a processing circuit comprising a processor coupled to a memory device storing instructions thereon that, when executed, cause the processing circuit to perform operations comprising:
acquiring an ultrasound image obtained using the transducer;
identifying, from a database, one or more similar images based on the ultrasound image, wherein the one or more similar images are identified based on depicting a same anatomical region or feature as the acquired ultrasound image; and
generating, using a generative artificial intelligence model, synthetic image data using the one or more similar images, such that the synthetic image data matches at least one characteristic of the ultrasound image.

2. The ultrasound imaging system of claim 1, wherein the operations further comprise establishing the database using a plurality of reference images.

3. The ultrasound imaging system of claim 2, wherein the plurality of reference images are retrieved from a plurality of image sources.

4. The ultrasound imaging system of claim 2, wherein the plurality of reference images correspond to a plurality of image qualities.

5. The ultrasound imaging system of claim 2, wherein the plurality of reference images are obtained using a plurality of imaging devices.

6. The ultrasound imaging system of claim 1, wherein the one or more similar images are identified by a self-supervised-learning model.

7. The ultrasound imaging system of claim 6, wherein identifying the one or more similar images further comprises:
training the self-supervised-learning model using the database;
extracting, using the self-supervised-learning model, the at least one characteristic of the ultrasound image and a plurality of characteristics from a plurality of reference images stored in the database; and
identifying, using the self-supervised-learning model, the one or more similar images based on a distance calculation between the at least one characteristic and each of the plurality of characteristics.

8. The ultrasound imaging system of claim 1, wherein the generative artificial intelligence model comprises a generative adversarial network.

9. The ultrasound imaging system of claim 1, wherein generating the synthetic image data further comprises:
training the generative artificial intelligence model using the database; and
transforming, using the generative artificial intelligence model, the one or more similar images to match the ultrasound image.

10. The ultrasound imaging system of claim 1, wherein the operations further comprise displaying the synthetic image data to a user via the display screen.

11. A system comprising:
a processing circuit comprising a processor coupled to a memory device storing instructions thereon that, when executed, cause the processing circuit to perform operations comprising:
acquiring a medical image obtained using an imaging device;
identifying, from a database, one or more similar images based on the medical image, wherein the one or more similar images are identified based on depicting a same anatomical region or feature as the acquired medical image; and
generating, using a generative artificial intelligence model, synthetic image data using the one or more similar images such that the synthetic image data matches at least one characteristic of the medical image.

12. The system of claim 11, wherein the system is an ultrasound imaging system comprising:
a transducer configured to transmit and receive an ultrasound signal, wherein the medical image is obtained by the transducer; and
a display screen.

13. The system of claim 12, wherein the operations further comprise displaying the synthetic image data to a user of the ultrasound imaging system via the display screen.

14. The system of claim 11, wherein the one or more similar images are identified by a self-supervised-learning model, and wherein identifying the one or more similar images further comprises:
training the self-supervised-learning model using the database;
extracting, using the self-supervised-learning model, the at least one characteristic of the medical image and a plurality of characteristics from a plurality of reference images stored in the database; and
identifying, using the self-supervised-learning model, the one or more similar images based on a distance calculation between the at least one characteristic and each of the plurality of characteristics.

15. The system of claim 11, wherein the generative artificial intelligence model comprises a generative adversarial network, wherein generating the synthetic image data further comprises:
training the generative adversarial network using the database; and
transforming, using the generative adversarial network, the one or more similar images to match the medical image.

16. A method comprising:
acquiring, by a processing circuit of an ultrasound imaging system, an ultrasound image obtained from a transducer of the ultrasound imaging system;
identifying, by the processing circuit and from a database, one or more similar images based on the ultrasound image, wherein the one or more similar images are identified based on depicting a same anatomical region or feature as the acquired ultrasound image;
generating, by the processing circuit and using a generative artificial intelligence model, synthetic image data using the one or more similar images, such that the synthetic image data matches at least one characteristic of the ultrasound image; and
displaying, by the processing circuit and via a display screen of the ultrasound imaging system, the synthetic image data to a user.

17. The method of claim 16, wherein the method further comprises establishing, by the processing circuit, the database using a plurality of reference images.

18. The method of claim 17, wherein the plurality of reference images are retrieved from a plurality of image sources, correspond to a plurality of image qualities, and are obtained using a plurality of imaging devices.

19. The method of claim 16, wherein the one or more similar images are identified by a self-supervised-learning model, and wherein identifying the one or more similar images further comprises:
training the self-supervised-learning model using the database;
extracting, using the self-supervised-learning model, the at least one characteristic of the ultrasound image and a plurality of characteristics from a plurality of reference images stored in the database; and
identifying, using the self-supervised-learning model, the one or more similar images based on a distance calculation between the at least one characteristic and each of the plurality of characteristics.

20. The method of claim 16, wherein the generative artificial intelligence model comprises a generative adversarial network, wherein generating the synthetic image data further comprises:
training the generative adversarial network using the database; and
transforming, using the generative adversarial network, the one or more similar images to match the ultrasound image.
